(19) United States
(12) Patent Application Publication
(10) Pub. No.: US 2009/0093692 A1
(43) Pub. Date: Apr. 9, 2009

(54) Methods and instruments for measuring tissue mechanical properties

(76) Inventor: Paul K. Hansma, Goleta, CA (US)

Correspondence Address: BERLINER & ASSOCIATES, 555 WEST FIFTH STREET, 31ST FLOOR, LOS ANGELES, CA, 90013, US

(21) Appl. No.: 12/228,940

(22) Filed: Aug. 18, 2008

Related U.S. Application Data
(60) Provisional application No. 60/965,623, filed on Aug. 20, 2007.

(51) Int. Cl. A61B 5/00; A61C 19/04; A61B 10/00

(52) U.S. Cl. 600/306; 433/72; 433/98

(57) ABSTRACT

Method and instrument for characterizing a material using a test probe constructed for insertion into the material, optionally with a reference probe constructed either for insertion into the material or to contact another material without insertion. The test probe is inserted at least a microdistance into the material (i) together with insertion of the reference probe into the material, (ii) with the reference probe contacting another material, or (iii) without a reference probe, and then withdrawn. The material property is determined by measuring an interaction of the test probe with the material, which may be related to the insertion of the test probe into the material, the movement of the test probe in the material, and/or the withdrawal of the test probe from the material.

### CROSS-REFERENCE TO RELATED APPLICATIONS

This application claims the benefit of Provisional Patent Application No. 60/965,623, filed Aug. 20, 2007.

### STATEMENT REGARDING FEDERALLY SPONSORED RESEARCH OR DEVELOPMENT

This invention was made with Government support under Grant no. RO1 GM 065354-05 from the National Institutes of Health. The Government has certain rights in this invention.

### FIELD OF THE INVENTION

The invention relates to an apparatus and method for measuring tissue mechanical properties.

### BACKGROUND OF THE INVENTION

U.S. patent application Ser. No. 11/417,494, filed May 3, 2006, titled Methods and Instruments for Assessing Bone Fracture Risk, and a U.S. Provisional Patent Application Ser. No. 60/921,788, titled Improved Methods and Instruments for Materials Testing, describe apparatuses and method for measuring material properties with particular application to the tissues bone and teeth. A paper by Hansma et al [1] related to the U.S. patent application Ser. No. 11/417,494 has also been published. The disclosures in U.S. patent application Ser. No. 11/417,494, and U.S. Provisional Patent Application Ser. No. 60/921,788, and the Hansma et al. paper [1] are all incorporated herein by reference. The devices described in the above referenced applications and paper and devices discussed in a book chapter [2] by Ottensmeyer et al. all depend on a reference probe on the surface of the material under test.

Another instrument for measuring soft tissue properties in living humans is the arthroscopic Scanning Force Microscope (SFM) designed to do nanometer scale measurements on the surface of cartilage as described in a paper [6]. The arthroscopic Scanning Force Microscope only probes on the nanometer length scale and it does not involve a reference probe that penetrates the cartilage tissue.

### BRIEF SUMMARY OF THE INVENTION

The present invention provides new and improved methods and instruments for measuring material parameters of tissues including soft tissues and teeth. These new methods and instruments are believed to significantly expand the range of materials that can be tested to include most, if not all, of the tissues of a human or animal, whether or not in the body, or in a plant. These tissues may include, but are not necessarily limited to, human and animal tissues such as connective tissues, fascia, joints, muscles, tendons, skin, vertebral disks, cartilage, and ligaments as well as groups of tissues including organs such as the liver, kidneys, pancreas, spleen, stomach, heart, brain, lungs, eye, uterus, bladder, and intestines. These tissues may also include, but are not necessarily limited to, plant organs such as roots, leaves, stems, flowers, seeds and fruits. Use of the invention enables the detection of tissue pathologies that are associated with changes in material properties including, but not limited to pathologies such as cancer, scarring, infection and necrosis that are inside tissues and organs.

More particularly, the invention provides a method and instrument for characterizing a material using a test probe constructed for insertion into the material, optionally with a reference probe constructed either for insertion into the material or to contact another material without insertion. The test probe is inserted at least a microdistance (i.e., at least one micron) into the material (i) together with insertion of the reference probe into the material, or (ii) with the reference probe contacting another material, or (iii) without a reference probe, and then withdrawn. In one embodiment a property of the test probe is measured related to its interaction with the material. In another embodiment, a property of the test probe is measured related to its insertion into the material. In yet another embodiment, a property of the test probe is measured related to its movement in the material, which may be part of the insertion of the test probe. In still another embodiment, a property of the test probe is measured related to its withdrawal from the material. In specific embodiments, the test probe is coated with a chemical or biological functionality to interact with the material. In other specific embodiments, the reference probe is in the form of a sheath in which the test probe is disposed, the end of the reference probe being proximal the test probe tip serving as said reference. In still other specific embodiments, the test probe is constructed at one end as a dental pick with the reference distant from the active region of the test probe.

### BRIEF DESCRIPTION OF THE DRAWINGS

For a more complete understanding of the present invention, reference is now made to the following descriptions of certain embodiments taken in conjunction with the accompanying drawings, in which:

FIG. 1 comprises FIGS. 1A-1B, with FIG. 1A representing our Prior Work in which a reference probe is in contact with the surface of the material under test and FIG. 1B schematically illustrating certain salient advances over our prior work (FIG. 1A);

FIG. 2 comprises FIGS. 2A-C, which respectively show three options for an optional stop to limit the penetration of a reference probe;

FIG. 3 is a cross-sectional view of a first embodiment of a measurement head;

FIG. 4 comprises FIGS. 4A-C, where FIG. 4A is a cross-sectional view of a second embodiment of a measurement head and FIGS. 4B and C show top views of an exemplary measurement system for this embodiment;

FIG. 5 comprises FIGS. 5A-D, where FIG. 5A is a cross-sectional view of the ends of a test probe and a reference probe in an embodiment especially suited to rotational measurements such as with the measurement heads of FIGS. 3 and 4, and where FIGS. 5B-D show cross-sections that are orthogonal to the cross section of FIG. 5A;

FIG. 6 is a cross-sectional view of a third embodiment of a measurement head;

FIG. 7 is a cross-sectional view of a fourth embodiment f a measurement head and test probe assembly C installed on a support fixture A holding a reference probe assembly B;

FIG. 8 comprises FIGS. 8A-C where FIGS. 8A and 8B are cross-sectional views of two embodiments of a measurement head intended for dental applications, and FIG. 8C shows how a right angled test probe that may be preferred for dental applications and some other applications inside body cavities can be driven by linear measurement heads such as shown in FIGS. 6 and 7;

FIG. 9 comprises FIGS. 9A and 9B, which are cross-sectional views of an embodiment of a measurement head adapted for dental applications;

FIG. 10 comprises FIGS. 10A and 10B, which show two examples of mechanisms to move the test probe in a linear fashion relative to a reference probe;

FIG. 11 is a generalized embodiment of the invention that can be used with indexed imaging to probe specific regions of specific tissues identified by that imaging. The Figure shows a cross-section of a patient's body with the test probe and reference probe placed in an internal organ; and

FIG. 12 comprises FIGS. 12A-C, which are detailed drawings of another version of the measurement head of a currently preferred embodiment of our invention.

### DETAILED DESCRIPTION OF CERTAIN EMBODIMENTS OF THE INVENTION

A common feature of all the described embodiments is a test probe that is inserted into a material under test to measure local properties of the material under test. In at least certain embodiments, the region of the material under test that is being probed can be determined by the position of a reference probe that shields the test probe from the material except where the test probe extends beyond the reference probe. Many of the described embodiments are particularly suitable for the study of non-mineralized or soft tissue in plants, animals or humans.

The various described embodiments may be used to facilitate the measurement of:

(a) a mechanical property of the material;

(b) the resistance of the material to motion of the test probe;

(c) a curve of the indentation depth into the material versus force needed;

(d) indentation of the material at a fixed force;

(e) indentation of the material at a fixed impact energy;

(f) the adhesion of the material on the test probe;

(g) the elastic modulus or viscoelastic properties of the material;

(h) the resistance of the material to creep or fatigue fracture;

(e) indentation of the material at a fixed impact energy;

(f) the adhesion of the material on the test probe;

(g) the elastic modulus of the material;

(h) the resistance of the material to fatigue fracture;

(i) the resistance to penetration of a screw into the material;

(j) the rotary friction on the material;

(k) a curve of the indentation depth vs. time after an impact;

(l) a curve of the force vs. time after impact to set distance;

(m) curves of the indentation depth vs. time for repetitive cycles or impacts;

(n) maximum indentation force;

(o) maximum indentation distance;

(p) energy dissipated during the indentation and retraction cycle or an impact;

(q) adhesion force during retraction;

(r) contact area of the test probe and sample;

(s) any combination of the above parameters;

(t) any change in those parameters, or combinations of those parameters, in multiple cycle testing; or

(u) the response of the material to a series or combination of the above measurements.

At least some embodiments of the present invention can also be used hand held by a physician or other person to probe properties such as listed above continuously or intermittently as the probe assembly, consisting of the test probe and reference probe, is moved through a tissue to probe for the changes in properties. This could be useful, for example, in probing for fibrosis or other anomalous regions in an organ and delineating their boundaries or for probing for boundaries and shapes of different types of tissues within an organ or other assembly of multiple tissues.

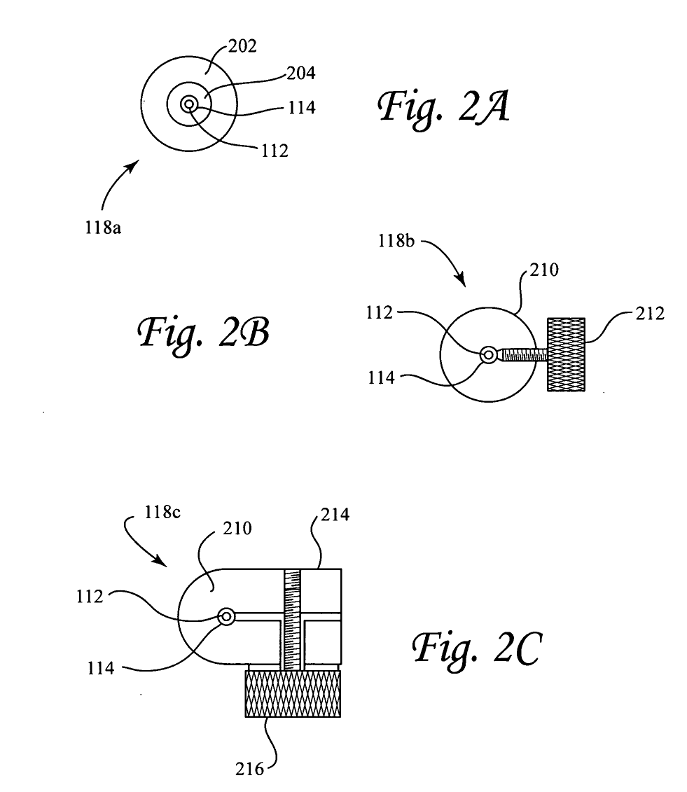

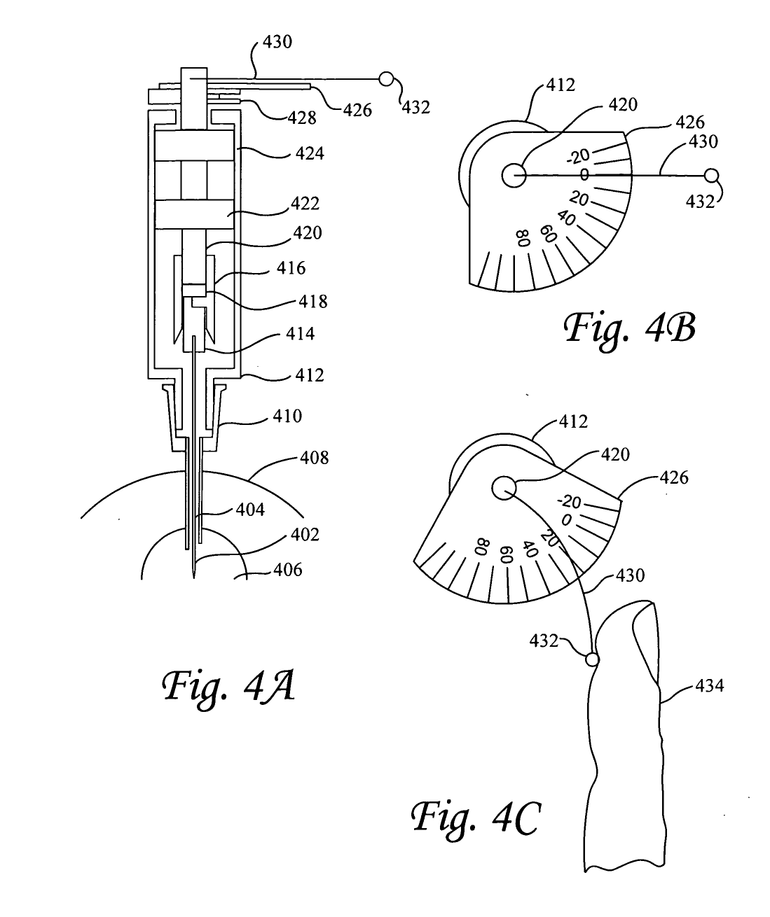

Many embodiments of the present invention can use much of the same hardware as described in U.S. patent application Ser. No. 12/079,444, specifically, the embodiments shown in that application as FIGS. 4, 5, 12, 13, 14, 15, and 24 as well as the embodiments shown as FIGS. 2, 3, 8, 12, 13, 14 and 17 of U.S. patent application Ser. No. 11/417,494, it being understood that the reference probe is not necessarily restricted to remain substantially on the surface of the material under test, but rather can be inserted into the material under test in some cases. For example, the reference probe can be inserted into a suspected carcinoma and the resistance of the material inside the carcinoma to motion of the test probe, which projects beyond the reference probe, can be measured. The test probe can be coated with appropriate coatings, such as antibodies bound to thiols on a gold tip or other chemical functionalities either bound to the tip with thiol or silane coupling or deposited as coatings with vacuum sublimation or evaporation or electroplated.

In other cases the reference probe can be on the surface of a first material while the test probe is making measurements on a second material. For example the reference probe can be on the surface of a patient's skin while the test probe was making measurements on the muscle below the skin. In fact, as described in more detail below, the reference probe can be optional in some cases. For example a dentist can insert a test probe into a suspect region on a patient's tooth and the force to remove it monitored by a dentist to distinguish fissures from caries. Even in this case, however, it may be useful to have a reference probe as a scale to measure the deflection of a test probe with an angled tip that is similar in design to current dental instruments used for probing fissures and caries. This scale can, for example, be at right angles to the wire of the test probe to measure the deflection of the test probe to measure the force, thereby making it possible for the dentist to get a quantitative measure of the applied testing force and of the adhesion force as the test probe is pulled away from the tooth.

In still other cases it is appropriate to provide two separate mechanisms to implement a reference probe having two separate and distinct functions: (1) to provide a reference position for measuring angular or linear displacements of the test probe and (2) to shield the test probe from contact with tissue that is not being probed, but which must be traversed to get to the tissue being probed. For example, it might be appropriate to have a tripod set on the surface of the skin to set the depth that is being probed while a tube over the test probe serves to isolate it from the tissue being traversed between the surface of the skin and the tissue being probed. In this case the tripod can clamp to the tube with a slideably adjustable clamp to adjust the depth.

For these new methods and instruments typically longer reference probes and test probes are needed. For example, to reach all parts of the interior of a muscle from the surface of the patient's skin the reference probe might need to be from 1 to 4 inches (2.5 to 19 cm) long depending on the location of the muscle and its size. The test probe might extend from 0.1 to 1 inch (2.5 to 25 mm) beyond the end of the reference probe. The trade off is that for longer extensions of the test probe beyond the end of the reference probe, the signal from viscoelastic interactions of the exposed part of the test probe with the region of the muscle under test will be larger. For shorter extensions of the test probe beyond the end of the reference probe, the signal from viscoelastic interactions of the exposed part of the test probe with the region of the muscle under test will be smaller, but give information with higher spatial resolution for precisely determining the position and boundaries of tissue with different mechanical properties due to necrosis or other conditions within the muscle. Similar trade offs between signal magnitude and special resolution will determine probe geometries for probing other tissues.

In addition to probe geometry and coating, other ways to increase specificity in measuring material properties include the way in which the test probe is moved relative to the reference probe and the tissue being probed. In accordance with the specific requirements of a particular application, the motion may be linear, linear oscillatory, rotational, and/or rotational oscillatory motion. In some cases the linear oscillatory motion, as detailed in U.S. patent application Ser. No. 11/417,494 and U.S. Provisional Patent Application Ser. No. 60/921,788, is useful because there is a theoretical basis for getting important material properties such as elastic modulus, creep and hardness in the case of indentations into a material and other methods for determining viscoelastic properties in viscoelastic materials. Rotational instruments are easy to produce in a convenient size to hand hold, with diameters in the range of 0.7 to 1.5 cm. Rotational oscillatory motion can be driven with an oscillating drive signal and the oscillating phase and amplitude can be detected. The phase may be of special interest in the case of test probes coated with antibodies or in other cases when the energy dissipation of the interaction between the test probe and the tissue is of interest. In particular embodiments, the signal is enhanced by micro patterning of the coating. For example, stripes of antibody coating, alternating with stripes of another coating or bare stripes, which are applied parallel to the axis of the test probe with angular widths of 30 degrees each (6 antibody and 6 other alternating stripes around the cylinder of the test probe), can be provided and are especially useful with rotational oscillations of order 1 to 30 degrees.

The interaction forces of antibody or other coated test probes with tissues are large enough to be easily measured. For example, measurement of single molecule interaction forces with an Atomic Force Microscope in the range of 10 to 300 pN are described in the Fantner et al. paper titled “Sacrificial bonds and hidden length dissipate energy as mineralized fibrils separate during bone fracture” [3].

With these interaction forces, we can make order of magnitude estimates of forces we might find when trying to rotate or translate a test probe that had bound to a tissue with many molecular bonds in parallel. Assuming a molecular density of one molecule per (10 nm)², an interaction force per molecule of 50 pN, a coated region 40 microns wide and 5 mm long on the test probe, and a fractional binding of 1% we would get a force of 50 pN/molecule×40 microns×5 mm×1 molecule/(10 nm)2×0.01=1 mN. If there were 20 such strips around a 0.18 mm diameter test probe, the total interaction force would be 20 mN, which is in the range of measurement. A complete coating of the tip would give 28 mN.

In experiments with prototype instruments the inventor has seen forces in the range of 0.2 to 10 Newtons in non-mineralized tissue with a test probe of diameter 0.375 mm and an exposed length beyond the reference probe of 10 mm operated at amplitudes of motion parallel to the test probe of 0.1 to 1 mm at frequencies of 1 to 10 Hertz. These forces are in a range that is easily measurable by commercial load cells from Futek, Transducer Technologies and other companies. These forces would create torques in the range of (0.2 to 10 Newtons)×0.187 mm=0.037 to 1.87×10⁻³ Newton meters. This is measurable at the low end of the ranges available with commercial torque sensors from various companies. Omega offers the model TQ202-25Z with a range of 0 to 0.175 Nm. Futek offers the Futek model FSH 02002 torque sensor with a range of 0 to 0.5 Nm. Practical instruments can benefit from custom torques sensors built with strain gauges from companies such as National Instruments and Omega. One advantage of building a custom sensor for torque or force is that this can facilitate both miniaturization and making a wireless instrument. For example, MicroStrain offers the Agile-Link wireless data acquisition system that can monitor and transmit results from multiple strain gauges thereby providing a readout system for strain gauges and a wireless transmission system at the same time. A simple mechanical system for measuring torques in the necessary range is also presented below.

One class of embodiments of the present invention involves modifying commercial viscometers, rheometers or micro rheometers, by adding a probe assembly. In general, the rotating test probe for tissue applications will be smaller in diameter than the standard probe of typical commercial viscometers. However, the stronger interaction of a test probe, especially an appropriately coated test probe, with tissue, compared to the weaker interaction of the standard probe with liquids that are typically tested helps compensate for the smaller diameter.

The probe assembly, consisting of the reference probe and the test probe together, can be as small as an acupuncture needle, even with commercially available torque and strain guages. Specifically, a 33 gauge syringe needle has an outer diameter of only 0.21 mm, which is within the range of diameters of acupuncture needles, 0.18 to 0.3 mm. In this case the test probe would need to be smaller than 0.11 mm in diameter, the inside diameter of a 33 gauge syringe. Wire this small is readily available for use in fabricating test probes: titanium or medically approved stainless steel is suitable for use in tissue. Alternately, a 27 or 28 gauge syringe with inner diameters of 0.21 and 0.18 mm respectively, can use a commercial small diameter acupuncture needle, 0.18 mm in diameter. The use of a probe assembly as small in diameter as an acupuncture needle allows testing of tissue without anesthesia, just as acupuncture is done without anesthesia. The trade off is that the signals of force or torque are smaller for smaller test probes. Further it is somewhat more difficult to fabricate test probes with complex shapes in small diameters. Test probes up to several mm in diameter can be used in some tissues based on the precedent of needle biopsies. In the case of larger probes, local anesthesia can be used.

The present invention can also be used for acupuncture. The precise movement of the test probe in a precise region, defined by the reference probe, which is allowed by the present invention, can be optimized for the therapeutic benefits of acupuncture therapy. Even without the reference probe, the precise movements allowed by the present invention can, in some cases, be therapeutically better than hand movements.

Microneedles with diameters of 0.1 mm are now being fabricated of titanium [4]. Microneedle technology can be useful in giving precisely shaped test or reference probes of small diameter.

The insertion and placement of the probe assembly of the instrument can be guided by imaging. Ultrasound imaging, for example, can be done as the probe assembly is being inserted in real time. MRI or CAT scans can map sites that needed investigation. In other cases, manual palpation might locate sites for investigation, such as for breast cancer diagnosis. Medical robots can insert the probe assembly to put the exposed, sensitive region of the test probe in sites mapped by imaging techniques.

Optically transparent test probes such as quartz or glass fibers can be used to get other material properties, such as spectroscopic information, from probed regions. Optical fibers are available commercially from, for example, Edmund Optics, with outer diameters as small as 0.14 mm, which would fit inside a 33 gauge syringe, which would have an outer diameter, 0.21 mm, in the range of acupuncture needles, 0.18 to 0.30 mm. These fibers have a fused silica core and can be used for both visible and UV spectroscopy. Raman spectroscopy, in particular, has shown promise for giving information about tissues [5]. This information might be enhanced by placement of the optically transparent test probe within the tissue as with the present invention. It might also be useful to correlate the spectroscopic information with mechanical measurements of local material properties by, for example, rotating or translating the test probe. Further, moving the test probe in and out of the reference probe can function like a chopper to alternate signal from the tissue with background signal. The background signal, from, for example florescence, can then be subtracted from the signal from the tissue to optimize sensitivity and linearity.

Certain embodiments of the present instrument can be combined with an endoscope to thereby provide information about the material properties of tissues visualized by the endoscope. They can be attached to the endoscope, integrated with the endoscope or operated independently from the endoscope.

For some applications it may be desirable to have a sheath or multiple sheaths covering the reference probe. These multiple sheaths can consist of multiple tubes that fit over a tubular reference probe. As an example of the use of such a system, a sheath that is sharpened like a hypodermic needle can aid tissue penetration, allowing the reference probe, which can be held inside the sheath during tissue penetration, to have a blunt end to better define the region of the test probe that is exposed beyond it during testing. A figure showing an embodiment of this embodiment of the invention is in U.S. patent application Ser. No. 11/417,494. This type probe can also be used for other tissues using the methods in this document. Similarly, various shaped test probes shown in U.S. patent application Ser. No. 11/417,494, and U.S. Provisional Patent Application Ser. No. 60/921,788 can also be used, among others, for other tissues using the methods of this invention.

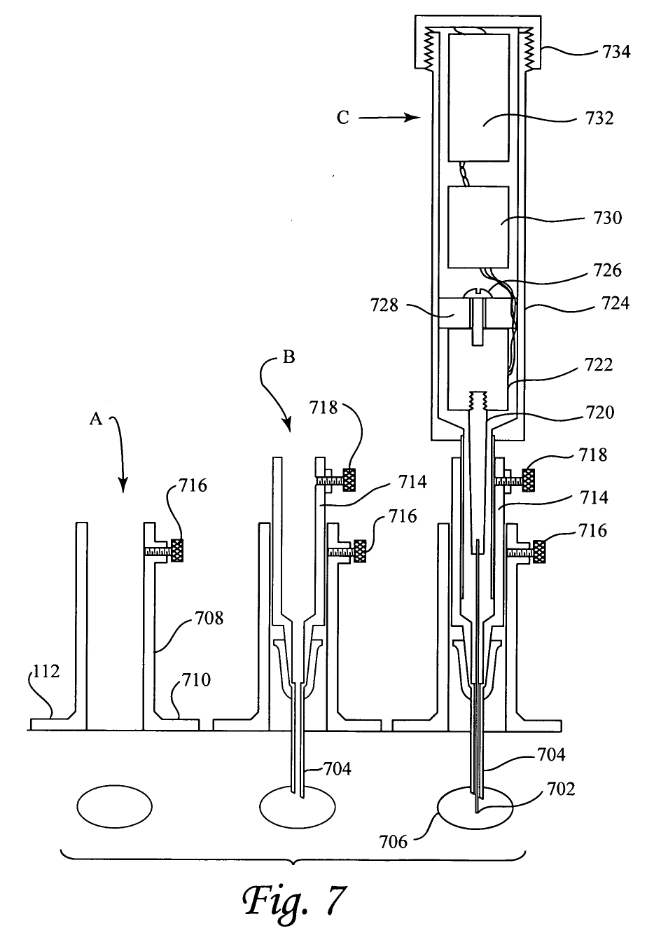

For other applications it may be desirable to have the reference probe closed at the end and have the test probe exposed to the tissue only through an opening cut in the side of the reference probe. This opening can, for example, be an oval that has a major axis that aligned with the axis of the reference probe and has a length from 0.1 to 10 mm and has a minor axis that is perpendicular to the axis of the reference probe and has a length from 0.05 to 1.0 times the diameter of the reference probe. The opening can also have a rectangular or other shape. This type of probe assembly can protect a fragile test probe from damage or contamination as tissue is being penetrated by the closed reference probe. After the tissue is penetrated to the desired depth such that the opening is positioned in the region of the tissue to be tested, then the test probe can be rotated or translated inside the reference probe and the interactions of the region of the test probe exposed through the opening with the tissue adjacent to the opening can be tested. Optionally, using a system like the one illustrated in FIG. 7 with the probe assembly described here, it is possible to keep the test probe out of the reference probe until the reference probe is in the desired position, with its opening positioned in the tissue to be tested. At that time the test probe can be inserted into the reference probe. This further minimizes the possible contamination of the test probe as the reference probe is inserted. It is also possible to use a reference probe that is open at the end with an opening in the side. If the test probe does not project beyond the opening at the end of the reference probe, the dominant interaction of the test probe with the tissue can still be with the tissue adjacent to the opening in the reference probe.

As stated above, the measurement head for measuring the forces and motions and/or the torques and rotations of the test probe relative to the reference probe can be as described previously in U.S. patent application Ser. No. 11/417,494, U.S. Provisional Patent Application Ser. No. 60/921,788, and the Hansma et al. paper [1] (hereafter “Prior Work”) Here, however, we will also show new measurement heads designed to be more compact and optimized for non-mineralized or soft tissue applications.

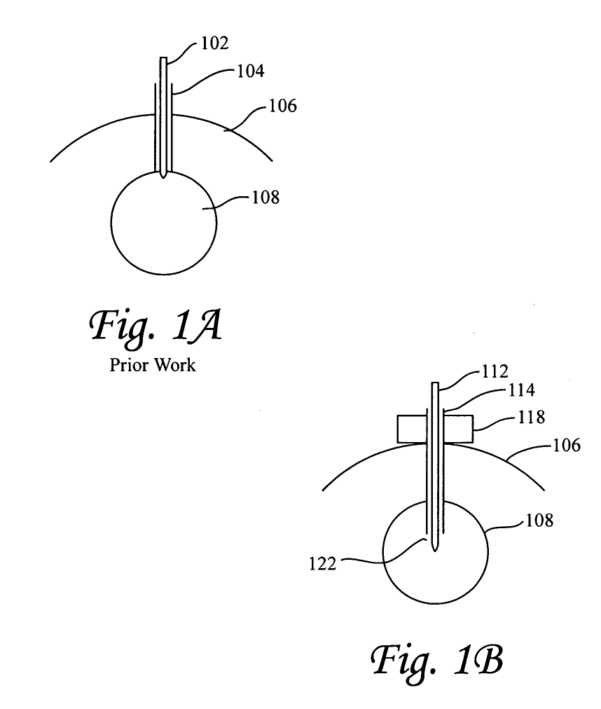

FIG. 1A shows that in our Prior Work the test probe 102 indents the surface of a material under test 108 a distance that is measured relative to a reference probe 104 which rests substantially on the surface of the material under test 108. The material under test 108 may be covered with another layer 106 that is penetrated by the reference probe 104 or the material under test 108 can be bare.

FIG. 1B shows that in certain embodiments of the present invention the test probe 112 can probe not only the surface of a material under test 108, but also the interior. The test probe 112 is sensitive to the material properties of the material under test 108 in the part 122 that projects beyond a reference probe 114 that also can penetrate the material under test. The translation or rotation of the test probe 112 is measured relative to the reference probe 114. The positioning of the probe assembly, consisting of the test probe 112 and the reference probe 114 can be determined by an optional stop 118 on the surface of a second material 120. This second material 106 might consist of the skin and underlying soft tissue covering a muscle as the material under test 108. The region of the test probe 122 that is exposed can be changed by moving the reference probe 114. In some cases it may be desirable to protect the test probe 112 from contact with tissue during insertion of the probe assembly by covering it with the reference probe. After the desired region to be probed is reached the reference probe 114 can be partially withdrawn exposing the end of the test probe 122. This may be desirable in cases where the test probe is fragile as for an optical fiber or in cases where it has coatings that should be protected until use.

FIG. 2A shows a specific embodiment 118a consisting of a rigid material 202 covering a soft elastic layer 204 that is held by friction on the reference probe 114 outside the test probe 208. FIG. 2B shows that the optional stop 118b can also consist of a rigid material 210 that has a set screw 212 that can clamp to the reference probe 206 outside the test probe 208. FIG. 2C shows that the optional stop 118c can also consist of a clamp 214 that is closed by a screw 216 onto the reference probe 114 outside the test probe 112. FIG. 2C is a preferred embodiment because it conveniently stays in place securely without risk of denting or damaging the reference probe 114.

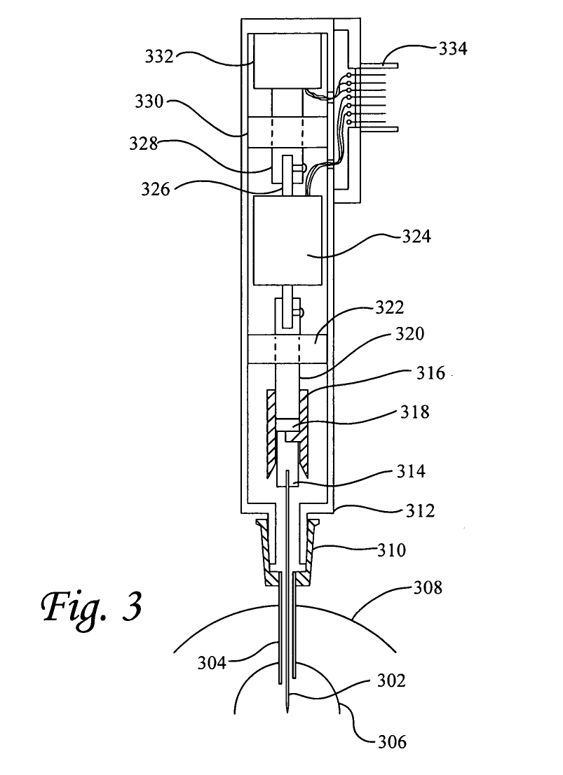

FIG. 3 shows a compact measurement head for measuring the torque produced by rotation of the test probe 302 relative to the reference probe 304 inside a tissue 306 covered by other tissues 308. The reference probe 304 is secured to the case of the measurement head 312 by a Leur fitting 310. Alternately the reference probe 304 can be secured to the measurement head 312 by a screw fitting, a friction fitting, a clamp or other means. The test probe 302 terminates at its upper end in a ferromagnetic plug 314 that is held in an indexing collar 316 against a magnet 318 connected to a shaft 320. The shaft 320 passes through a low friction bearing 322 such as a ball bearing and is connected at the other end to one end of the shaft 326 of a motor 324, which can be a stepping motor or a simple motor. The shaft of the motor 326 is connected at the other end to a shaft that goes through another low friction bearing 330 and terminates in a torque sensor 332. The electrical leads for the motor 324 and the torque sensor 332 pass out of the case 312 on a multi pin electrical connector 334. In operation, the torque produced by the interaction of the test probe 302 with the tissue 306 as the test probe is rotated in a continuous, intermittent or oscillatory motion by the motor 324 is measured.

FIG. 4A shows another compact measurement head for measuring the torque produced by rotation of the test probe 402 relative to the reference probe 404 inside a tissue 406 covered by other tissues 408. The reference probe 404 is secured to the case of the measurement head 412 by a Leur fitting 410. As will also be true for other embodiments, alternately the reference probe 404 can be secured to the measurement head 412 by a screw fitting, a friction fitting, a clamp or other means. The test probe 402 terminates in a ferromagnetic plug 414 that is held in an indexing collar 416 against a magnet 418 connected to a shaft 420. The shaft 420 passes through a low friction bearing 422 and then another low friction bearing 424 and is connected at the other end to an angular scale 426 held on the shaft 420 by a collar 428 with a set screw. It is also connected to a fine wire 430 that terminates in a ball 432. FIG. 4B shows a top view of the scale 426 and the fine wire 430. In operation, as shown in FIG. 4C the ball 432 is pressed with a finger 434 to apply a torque to the shaft 420. The amount of torque can be read off the scale 426, which is shown calibrated in degrees. These degrees can be converted to torque by connecting the shaft to a calibrated torque sensor. This calibration will be a function of the diameter of the wire 430. In practice we have found stainless steel or spring steel wires with diameters in the range of 0.003 to 0.010 inches (0.08 to 0.25 mm) to provide suitable torques for measurement in soft tissue.

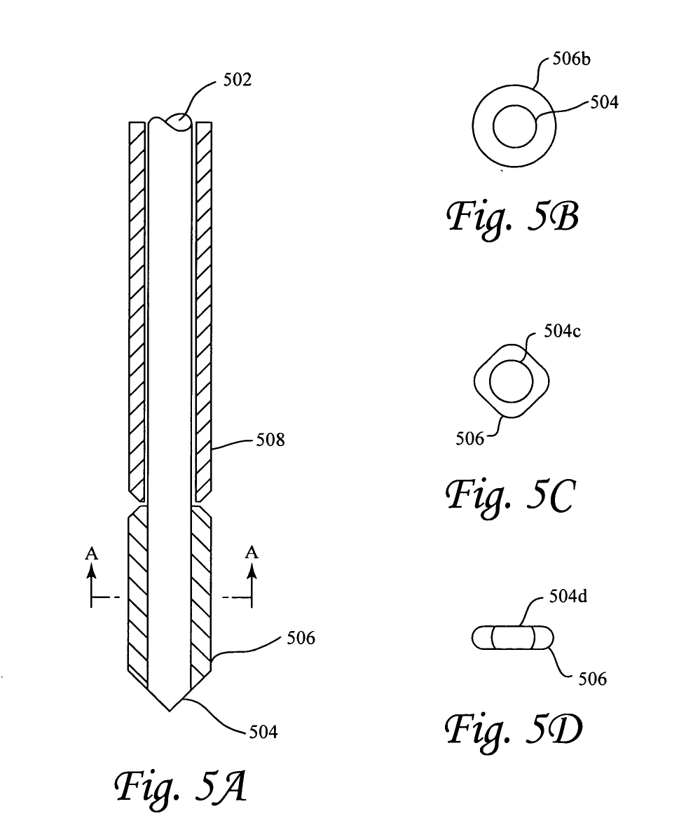

FIG. 5A shows a different geometry for the end of the test probe 502 that is suitable for the measurement heads shown in FIGS. 3 and 4 as well as other measurement heads that involve rotations rather than translations. In this case the end of the test probe 504 can be enlarged to the outer diameter of the reference probe 508 by adding material 506. This extra material can be cylindrical 506b as shown in FIG. 5B, a square with rounded corners 506c as shown in FIG. 5C, or have an elongated shape 506d as shown in FIG. 5D. Other possibilities are elliptical or eccentric like a cam. In general the greater the departure from cylindrical the larger the torque signal for a given rotation but more potential for tissue damage. This same general principle also applies to test probes used with linear motion.

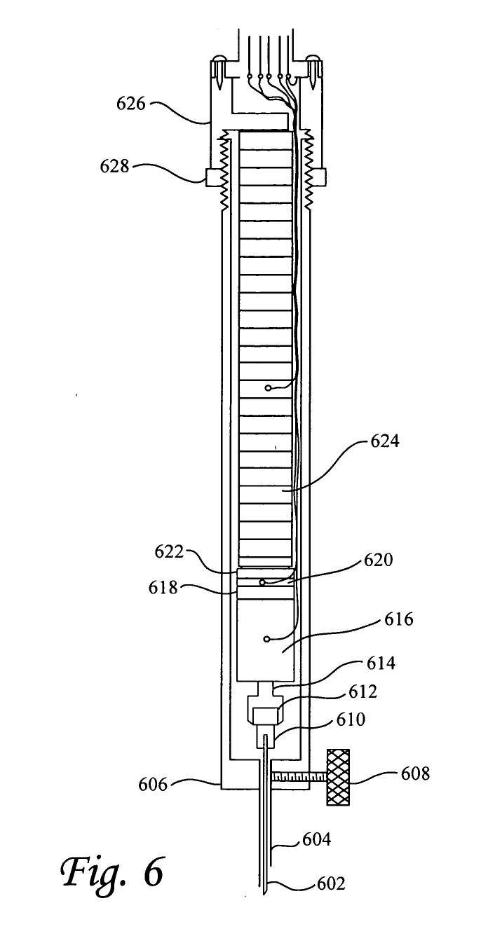

FIG. 6 shows a compact measurement head designed to measure the forces produced by translations of the test probe 602 relative to the reference probe 604. In this case the reference probe 604 is held in the case 606 by a screw 608. The test probe 602 terminates at its upper end in a ferromagnetic cylinder 610 that is removably mounted to a magnet 612 held in a shaft 614. This shaft 614 is connected to a load cell 616 to measure force. The load cell 616 is connected to an insulator 618, an electrical shield 620, another insulator 622 and a piezoelectric stack 624. This piezoelectric stack 624 can, for example be a multilayer piezoelectric actuator such as the Tokin model AE0203D44H40, which can produce a 40 micron displacement. The other end of the piezoelectric stack 624 is connected to an end cap 626 that is screwed onto the case 606 and held in place by a threaded locking ring 628. The amount that the test probe 602 projects beyond the reference probe 604 can be adjusted by loosening the screw 608 and sliding the reference probe 604 relative to the case 606 and then retightening the screw 608 or by loosening the locking ring 628 and rotating the threaded end cap 626 relative to the case 606 and then retightening the locking ring 628. This embodiment is particularly well suited to measurements at a wide range of frequencies because the piezoelectric stack 624, even when loaded with the extra mass of the components 602, 610, 612, 614, 616, 618, 620 and 622, can have a resonant frequency above 10 kHz.

FIG. 7 shows another measurement head with novel fixtures for adjusting the position of the test probe 702 and the reference probe 704 in the tissue of interest 706. First (Step A) a fixture 708 is placed on the skin of a patient or surface of a plant. It can, optionally, be positioned based on information from MRI or other imaging. It can be held in place with tape (not shown) over tabs 710 and 712 that project out from the fixture. Next (Step B) a reference probe assembly consisting of a reference probe 704 and a reference probe holder 714 is inserted into the fixture 708 to a depth, optionally, determined by imaging or other techniques. A locking screw 716 can hold the reference probe holder in the desired position. Next (Step C) the test probe 702 can be inserted into the tissue to a depth determined by imaging or other techniques. A locking screw 718 can hold the test probe holder 724 in the desired position. After measurement in one position the entire test probe—reference probe assembly can be moved to a new position by loosening the locking screw 716 while keeping the locking screw 718 fixed. Alternately the amount of projection of the test probe 702 for a given position of the reference probe 704 can be changed by loosening the locking screw 718 while keeping the locking screw 716 fixed. This system can be used for many different measurement heads such as the ones shown in previous figures and in Prior Work

The Step C portion of FIG. 7 also shows a new measurement head for manual, wireless operation. The test probe 702 is held in a shaft 720 that is connected to a load cell 722 (this can alternatively be a torque sensor for rotary motions). The load cell 722 is connected to the case 724 by a screw 726 through a plug 728 connected to the case 724. The leads from the strain gauges in the load cell, or torque sensor, 722 go through the plug 728 to a strain gauge readout circuit and wireless transmitter 730, for example by using the MicroStrain Agile-Link wireless data acquisition system referred to above. The unit is powered by a battery 732 that is accessible through the threaded cap 734. In use, either locking screw 716 or locking screw 718 is loosened and the case 724 is translated or rotated by hand and the resultant forces or torques, respectively, are monitored.

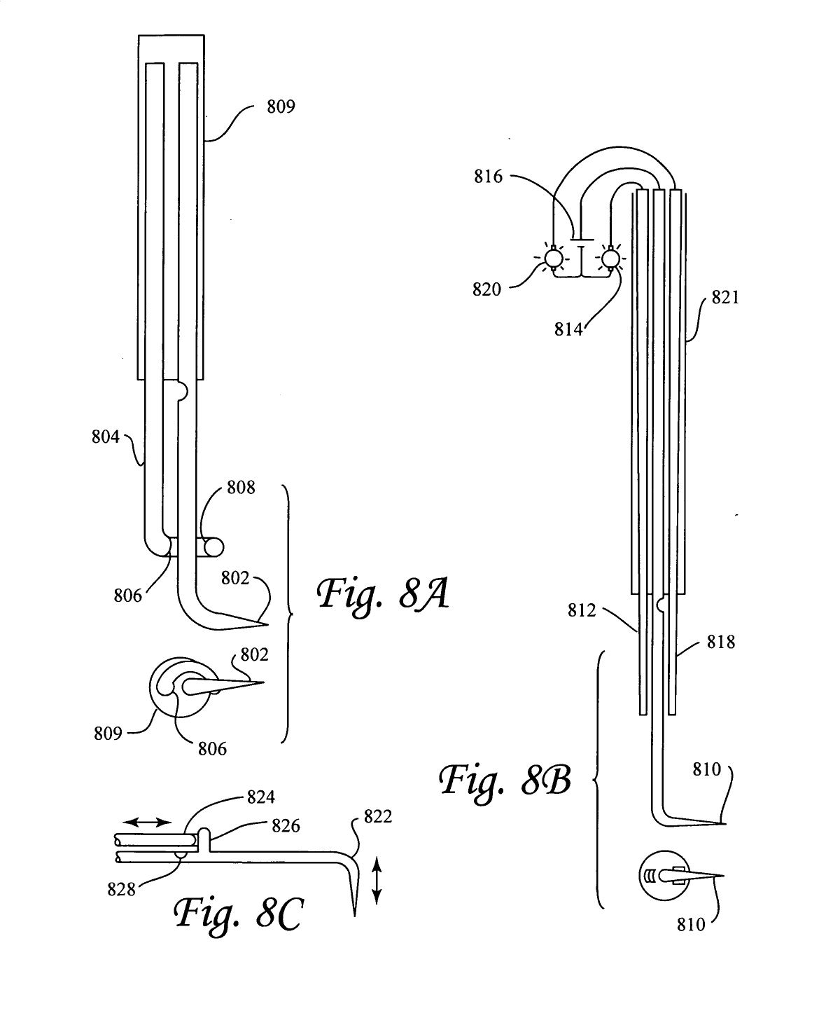

FIG. 8A-C shows three embodiments for dentistry that are designed to distinguish between caries and fissures by the difference in adhesion on withdrawal of a test probe that has been pressed against a tooth with a standard force. In FIG. 8A the test probe 802 is pressed against the tooth until it deflects and contacts the reference probe 804 at point 806. The confirmation can be the audible click on contact or the feel of the contact with the dentist's hand. Then the test probe 802 is withdrawn and it is determined if the withdrawal force is sufficient to make contact with the reference probe 804 at contact point 808. If the force is sufficient, the diagnosis is caries, if not, fissure. The test probe 802 and the reference probe 804 are mounted in a handle 809.

FIG. 8B shows an embodiment with an electrical readout. Contact of the test probe 810 with one piece of the reference probe 812 indicates that the correct test force has been achieved. This contact lights an electrical lamp 814 that is powered by a battery 816. On withdrawal the contact of the test probe 810 with the other piece of the reference probe 818, which indicates caries, is revealed by the lighting of another lamp 820, preferably of a different color. The lamps and battery can be incorporated into the handle 821. Audible sounds on contact may be preferred by some dentists and can be sounded together with the lamps lighting or without.

FIG. 8C shows that the right angled test probe 822 preferred for dental applications and some other applications inside body cavities can be driven by linear measurement heads such as shown in this application as FIGS. 6 and 7 as well as others in our Prior Work The shaft 824, which in the previous drawings of measurement heads was connected in a linear fashion to a test probe, can instead press on a lever 826 connected to the test probe 822. One advantage of this scheme is that it magnifies the motion of the shaft 824, which may be desirable when the shaft is driven by piezoelectric elements as in FIG. 6 since these piezoelectric elements have limited range. One disadvantage of this scheme is that the force to deflect the flexure 828 is added to the force between the test probe and the material under test and must be corrected for by subtracting the force to deflect the flexure.

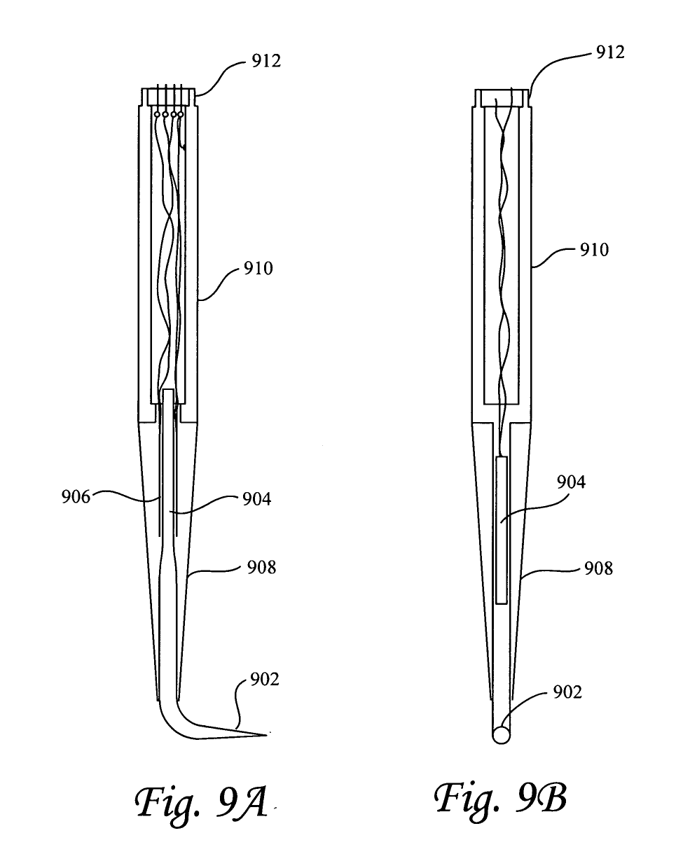

FIGS. 9A and 9B show two cross sectional views of an embodiment with a continuous readout. The test probe 902 has two strain gauges 904 and 906 attached to a flattened region. The region with the strain gauges is covered in a soft rubber or plastic sheath 908 to protect the strain gauges from fluids, for example saliva and water used for rinsing during dentistry. Wires from the strain gauges 904 and 906 run through the handle 910 to an electrical connector 912. The strain gauges 904 and 906 can, optionally, be operated in an A-B mode to increase signal and partially compensate for temperature changes. This connector 912 can be used with a cable to remote electronics or to a wireless transmitter and battery to monitor and transmit results from the strain gauges 904 and 906 to remote electronics, for example by using the MicroStrain Agile-Link wireless data acquisition system referred to above. The soft rubber or plastic sheath will add a component to the measured force and must be corrected for by the electronics by subtraction. It is also possible to build a compact electronics package that mounts directly to the connector 912. This electronics package or the remote electronics can include threshold detectors for the applied force and the withdrawal force that drive audible sounds or light signals or other indications that the correct applied force is reached and whether or not the withdrawal force is large enough for a diagnosis of caries in dental applications.

Other dental applications of embodiments of this invention involve evaluating the condition of gums and other mouth tissue as well as cavities revealed by X-rays, for example between the bottom of the root of a tooth that has been root canalled and the underlying bone.

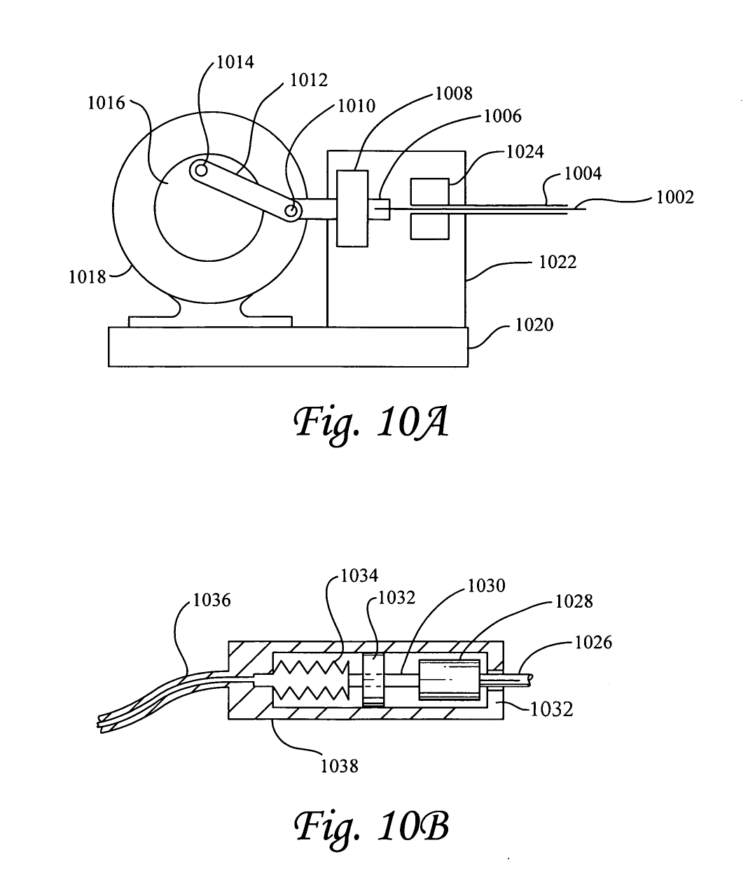

FIGS. 10A and 10B show two examples of mechanisms to move the test probe in a linear fashion relative to a reference probe. In FIG. 10A a Bowden cable consisting of an inner wire 1002 that will be connected to the test probe (usually via a load cell) and an outer sheath 1004 that will be connected to the reference probe. The inner wire 1002 is connected to a shaft 1006 that runs through bearings 1008 and is connected via a pin 1010 to a shaft 1012 that is connected via a pin 1014 to a disk 1016 that is rotated by a motor 1018. This motor 1018 can include a gear box and run either at constant speed or have adjustable speed. The motor 1018 can also be a stepping motor for more complex motions such as oscillations that drive the inner wire 1002 through an oscillatory motion of adjustable amplitude. The motor 1018 is mounted on a base 1020. A support 1022 for the bearings 1008 and a holder 1024 for the outer sheath 1004 is mounted on the base 1020. The holder 1024 can include a locking screw adjustment (not shown) to adjust the relative position of the outer sheath 1004 and the inner wire 1002.

FIG. 10B shows a hydraulic mechanism to move the test probe in a linear fashion relative to a reference probe. A shaft 1026 that will be connected to a test probe is connected at its other end to a load cell 1028 that is connected to a shaft 1030 that is supported by linear bearings 1032 and connects to bellows 1034. This bellows 1034 is connected to a hydraulic line 1036 that connects to a remote hydraulic pump (not shown) such as an axial piston pump or a simple piston pump or a pump run by a stepping motor for complex motions. The hydraulic line 1036, the bellows 1034, and the bearings 1032 are mounted in a case 1038 that is connected to the reference probe (not shown) by means such as displayed in FIGS. 3, 4, 6 and 7 or in references 6 and 7. Similarly the shaft 1026 can be connected to the test probe (not shown) by means such as displayed in FIGS. 3, 4, 6 and 7 or in Prior Work.

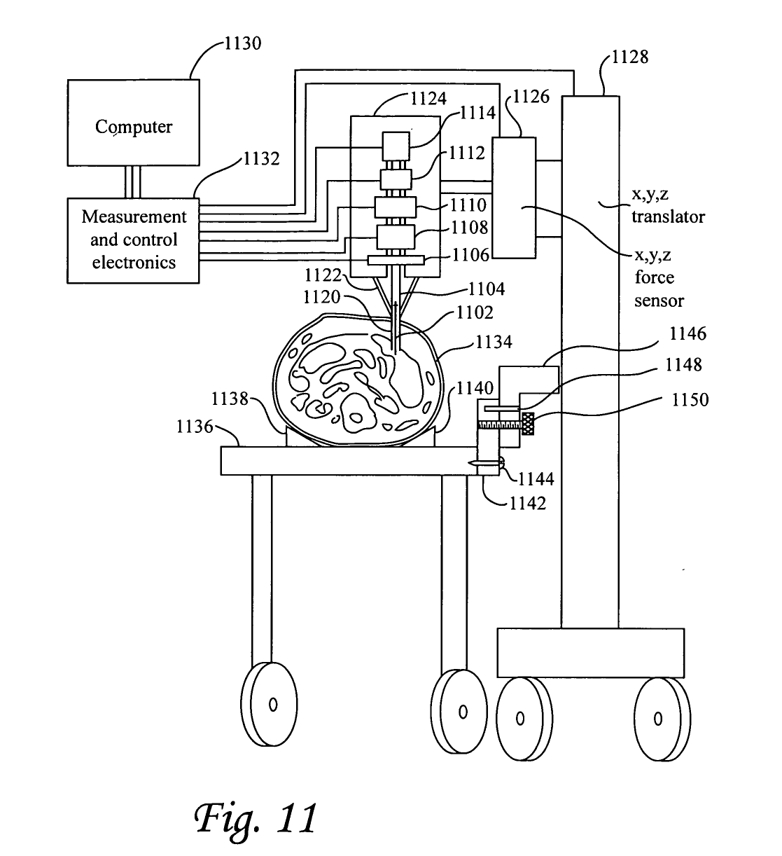

It will be appreciated that the previously described embodiments of the invention are for the most part relatively compact and can be easily hand held and inserted into the patient, animal or plant by hand. A larger and more versatile embodiment, in some ways similar to one known from our Prior Work is shown in FIG. 11.

The test probe 1102 is connected to a shaft 1104 that is connected to an optional torque and angular displacement sensor 1106 then to an optional torque generator 1108, then to an optional linear displacement sensor 1110, then to an optional force sensor 1112, and finally to an optional force generator 1114. The reference probe 1120 is connected via reference probe holder 1122 to the housing 1124 that holds the transducers and generators. The housing 1124 can be supported and positioned on the sample under test by a support that includes an optional x,y z force sensor 1126 and an optional x,y,z translator 1128. The positioning of the housing by the x,y,z translator 1128 can be controlled by a computer 1130 that also records signals for the x,y,z force sensor 1126 and the optional linear displacement sensor 1110 and the optional torque and angular displacement sensor 1106 via measurement and control electronics 1132. The computer 1130 also controls the optional torque generator 1108 and the optional force generator 1114 through the measurement and control electronics 1132. The computer can run Labview or similar programs to do the programmed control, data acquisition and data analysis as well as recording pertinent data about the tissue being tested such as patient name and type of tissue.

FIG. 11 also shows the equipment necessary for obtaining data from a location identified by imaging such as MRI or CAT. The patient 1134 is positioned on a table 1136 by indexing supports such as 1138 and 1140 and then imaged. It may be necessary to have the patient on a platform that is indexed in position onto the table 1136 if the table itself is not suitable for the imaging. After imaging, the table 1136 is attached via an indexing system consisting of a bracket 1142 that is attached with screws 1144 and optional indexing pins (not shown) to the table 1136 and another bracket 1146 that is attached to the x,y,z translator 1128 with optional indexing pins 1148 and screws 1150. The purpose of all the indexing is to enable positioning of the test probe 1102 and the reference probe 1120, under computer 1130 control to locations identified by analysis of imaging to be locations of interest in diagnosis. In operation the imaging is done first. Then the images are analyzed to identify locations of interest. The x,y,z coordinates of these regions of interest are transferred to the computer 1130. Then the x,y,z translator 1128 is moved into position and the computer 1130 positions the test probe 1102 and the reference probe 1120 at the identified locations of interest for measuring materials properties. Alternately, one can have the computer 1130 position the test probe 1102 at the correct lateral, x,y, position above the insertion site and then have a physician lower the instrument by hand, thus feeling the tissue and using his judgment and skill for the correct insertion speed and force until the desired point z is reached, as indicated by a display that the physician can see during insertion or by an audible signal. For this case it is useful to have a linear slide in the z direction to maintain the correct lateral, x,y, position during insertion.

This system can also be used to obtain biopsy samples by withdrawing the test probe 1102 into the reference probe 1120 before or after tissue penetration. This system can also be used to obtain spatially localized spectroscopic imaging if the test probe 1102 is an optical fiber and if that optical fiber is connected at its end to a fiber optic spectrometer (not shown). Many fiber optic spectrometers suitable for this purpose are available commercially such as the USB 4000 spectrometer configured for optimal light collection together with a HL 2000 light source and a bifurcated fiber with custom jacket to transmit the incident light from the HL 2000 light source to the location of interest and to collect the reflected or fluorescent light that results from the incident light for analysis by the USB 4000 spectrometer. In this case the optical fiber test probe 1102 can be optically coupled to another optical fiber inside the shaft 1104 and then routed out of the shaft 1104 and case 1124 at a convenient location and connected to the spectrometer and light source (not shown).

This same system as shown in FIG. 11 can also be used to combine real time ultrasound imaging with the measurement of materials properties. In this case the imaging can be done simultaneously with the materials property measurements and the indexing would not be necessary. The test probe 1102 and reference probe 1120 can be guided to the location of interest by watching it in the ultrasound image.

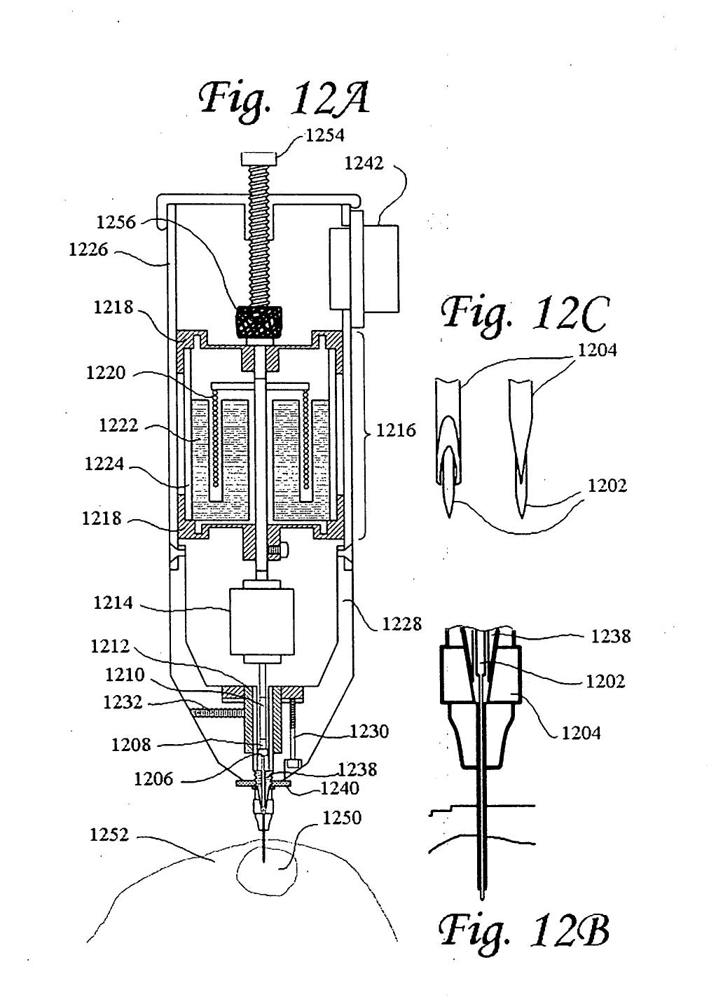

FIG. 12 is a detailed drawing of another version of the measurement head of a currently preferred embodiment of our invention. This version is designed to be used as Tissue Diagnostic Instrument (It is adapted for this use from a device described in U.S. patent application Ser. No. 12/079,444 for probing the surface of cartilage, skin or other tissues. In this version the test probe 1202 is a sharpened magnetic stainless steel rod that is of a diameter 0.009″ where it goes through the needle of the reference probe 1204, which is a specially sharpened 25 gauge hypodermic needle. At the other end, the test probe diameter is 0.062″ where it attaches to a permanent magnet 1206 that is attached to a shaft 1208 that connects to the core 1210 of an LVDT 1212 (for example Measurement Specialties MHR 025). This in turn connects to a load cell 1214 (for example the Futek LSB 200 or the Sensotec Model 34 precision miniature load cell) and then to a force generator 1216 consisting of two flexures together with a voice coil actuator (a modified version of BEI Kimco Magnetics LA16-27-000A) which consists of a moving coil 1220 in a magnetic field assembly 1222.

The flexure is described in more detail in U.S. patent application Ser. No. 12/079,444 and is included to guide the motion of the force generator and ensure that there is no substantial off-axis motion. The flexures design consists of a large, horizontal, thin inner membrane connected to an outer, thin, horizontal membrane through a vertical ring. The force generator 1216 is anchored in an inner shell 1224 that is capped by the flexures 1218. The force generator 1216 is held in an outer shell 1226. The outer shell is connected to a nose piece 1228, which supports the LVDT body 1212. The position of the LVDT body 1212 can be adjusted to zero or otherwise adjust the signal from the LVDT 1210, 1212 with a fine screw 1230 and is locked into place with set screws 1232. The nose piece 1228 also rigidly supports the reference probe 1204. The reference probe 1204 mates to a male Leur fitting 1238 that is threaded into the nose piece 1228 and held rigidly in position with a knurled locking nut 1240.

The electrical signals to actuate the force generator 1216 as well as the force signal from the load cell 1214 and the distance signal from the LVDT 1210, 1212 pass through an electrical connector 1242 (AMP 28 pin connector). The connecting wires are not shown for clarity.

In operation the reference probe 1204 is inserted into the tissue 1250 under test. Optionally the tissue 1250 may be covered with a layer of other tissue 1252, which may include skin.

The TDI prototype shown here has an optionally, adjustable compliance. The screw 1254 is used for adjusting the compliance of the force generator. This screw presses against a piece 1256 of rubber, Sorbothane, gum rubber or other elastomer that rests on the suspension of the force generator 1218 and can increase the effective spring constant of the suspension. Viscoelastic materials such as Sorbothane give better damping of oscillations, but have more non-linearity. In the limit that the screw is backed off the effective spring constant of the suspension (the compliance) is approximately 0.005 N/micron. It is used like that for hard tissues. In this case the compliance of the suspension is much smaller than the effective spring constant of the hard tissue and the force generated by the force generator is nearly the same as the force applied to the hard tissue (the TDI is approximately force controlled).

Perhaps ironically, it is desirable to stiffen the suspension for soft tissues. The point is that for soft tissues the compliance of the suspension is no longer much smaller than the effective spring constant of the soft tissue. Since it would be very difficult to fabricate a softer suspension, we take the opposite approach and stiffen the suspension (by compressing the block of rubber or other elastomeric material 1256 against the top of the suspension 1218) until the stiffness of the suspension is greater than the stiffness of the tissue.

FIG. 12B is a close up of the test probe 1202 and the reference probe 1204. The reference probe 1204 described here is made from a 25 gauge hypodermic needle. Other reference probes have ranged in size from as small as 30 gauge to as large as 14 gauge. The advantages of smaller diameter include smaller tissue damage and the potential of use on patients without anesthesia as in acupuncture. The advantages of larger diameter include more rigidity and more ability to accommodate complex test probes such as coated test probes.

The TDI could measure the interaction forces between antibody coated test probes and tissues. This would allow measurements of single molecule interactions as is currently achieved with an atomic force microscope [7]. Rupture forces in the range of 20 to 140 pN have been measured for many receptor-ligand interactions with Single-cell force spectroscopy [8]. With these interaction forces, we can make order of magnitude estimates of forces we might find when trying to rotate or translate a test probe that had bound to a tissue with many molecular bonds in parallel. Assuming a molecular density of one molecule per 10 nm², an interaction force per molecule of 50 pN, a coated region of area 4×10⁻⁶ m² (the exposed area of a probe that has been tested) and a fractional binding of 1% we would get a force of 50 pN/molecule×4×10⁻⁶ m²×1 molecule/10 nm²×0.01=200 mN. The current lower limits of sensitivity of the TDI for forces come from the friction between the test probe and the reference probe, of order 10 mN, and from the force noise in our force transducer, of order 5 mN in a 1 kHz bandwidth. Thus forces of the magnitude that could be expected from molecular interactions with coated tips should be measurable. The big problem would be non-specific interaction masking specific interactions. One approach to overcoming this masking effect would be to use a test probe coated on just one side that was exposed to the tissue under test though a window in the wall of a closed-end reference probe. The difference in the forces between the test probe and the tissue under test for the coated vs. uncoated side could be measured. This could naturally be extended with multiple coatings on multiple strips on the test probe, each exposed one by one through a slit in the wall of the closed-end reference probe.

It is important to note that though the present instrument is able to make basic measurements in a wide range of tissues (almost all tissues in the human body from very soft breast tissue to hard, mineralized tissues), more user convenience features, such as wireless operation, are possible. Specialized instruments for specific measurements in specific tissues could be developed at a small fraction of the cost of the fully versatile instrument. For example, for soft tissue, the force generator and distance transducer could be replaced with a simple motor that moved the test probe up and down a fixed amount independent of the nature of the tissue as long as the tissue was soft compared to the rigidity of the motor drive system. Only the force would need to be measured since the displacements would be known. The magnitude of the oscillating force could be read with a simple meter or indicator lights as a measure of the stiffness of the tissue.

FIG. 12C is a close up of the ends of the test probe 1202 and the reference probe 1204. The double beveled end on the reference probe 1204 is designed to minimize soft tissue entering the space between the test probe 1202 and the reference probe 1204. Soft tissue entering that space is also inhibited by slightly bending the ends of the reference probe 1204 toward the test probe 1202 as shown. There is space between the test probe 1202 and the reference probe 1204, typically a few thousands of an inch, to minimize friction. If this space becomes filled with soft tissue the friction goes up. The soft tissue can be removed by taking the reference probe 1204 off the head and flushing it with water or solvents and/or running a piece of wire that is close to the inside diameter through it to push out the tissue. Alternately the reference probe or test probe/reference probe assembly can be replaced.

### REFERENCES

The following references are each incorporated herein by reference.
1. Hansma, Paul K., Turner, Patricia J., and Fantner, Georg E.: Bone Diagnostic Instrument. Rev. Sci. Instr. 77, 075105 (2006).
2. Ottensmeyer, Mark P., Salisbury, J. Kenneth: In Vivo Data Acquisition Instrument for Solid Organ Mechanical Property Measurement, In a book titled Medical Image Computing and Computer-Assisted Intervention—MICCAI 2001: 4th International Conference Utrecht, The Netherlands, Oct. 14-17, 2001, Proceedings edited by Niessen, W., and Viergever, p. 975-982, M. Springer Berlin/Heidelberg
3. Fantner, Georg E., Hassenkam, Tue, Kindt, Johannes H., Weaver, James C., Birkedal, Henrik, Pechenik, Leonid, Cutroni, Jacqueline A., Cidade, Geraldo, A. G., Stucky, Galen D., Morse, Daniel E. and Hansma. Paul K.: Sacrificial bonds and hidden length dissipate energy as mineralized fibrils separate during bone fracture. Nature Materials 4, 612 (2005).
4. Parker, E. R., Rao, M. P., Turner K. L., Meinhart, C. D., and MacDonald, N.C.: Bulk Micromachined Titanium Microneedle. Journal of Microelectromechanical Systems 16, 289 (2007).
5. Timlin, Jerilyn A., Carden, Angela, Morris, Michael D., Rajachar, Rupak M. and Kohn, David H.: Raman Spectroscopic Imaging Markers for Fatigue-Related Microdamage in Bovine Bone. Anal. Chem. 72, 2229, (2000).
6. Staufer, U., Akiyama, T., Gullo, M.R., Han, A., Imer, R., de Rooij, N. F., Aebi, A. Engel, A., Frederix, P. L. T. M., Stolz, M., Friederich, N. Wirz, D.: Micro- and nanosystems for biology and medicine. Microelectronic Engineering 84, 1681, (2007).
7. M. Rief, F. Oesterhelt, B. Heymann, H. E. Gaub, Science 275, 1295 (February, 1997).
8. J. Helenius, C. P. Heisenberg, H. E. Gaub, D. J. Muller, Journal of Cell Science 121, 1785 (Jun. 1, 2008).

1. A method for characterizing a material using a test probe constructed for insertion into the material, optionally with a reference probe constructed either for insertion into the material or to contact another material without insertion, comprising:
inserting the test probe at least a micron into the material (i) together with insertion of the reference probe into the material, (ii) with the reference probe contacting another material, or (iii) without a reference probe;
moving the test probe in the material, which may be part of the insertion of the test probe;
withdrawing the test probe from the material; and
measuring a property of the material related to the test probe's (a) interaction with the material, (b) insertion into the material, (c) movement in the material, or (d) withdrawal from the material.

2. The method of claim 1 in which the material includes tissues in a human or animal body or in a plant.

3. The method of claim 1 in which the material property is one or more of:
(a) a mechanical property;
(b) the resistance of the material to microscopic fracture by the test probe;
(c) a curve of the indentation depth into the material versus force needed;
(d) indentation of the material at a fixed force;
(e) indentation of the material at a fixed impact energy;
(f) the adhesion of the material on the test probe;
(g) the elastic modulus of the material;
(h) the resistance of the material to fatigue fracture;
(i) the resistance to penetration of a screw into the material;
(j) the rotary friction on the material;
(k) a curve of the indentation depth vs. time after an impact;
(l) a curve of the force vs. time after impact to set distance;
(m) curves of the indentation depth vs. time for repetitive cycles or impacts;
(n) maximum indentation force;
(o) maximum indentation distance;
(p) energy dissipated during the indentation and retraction cycle or an impact;
(q) adhesion force during retraction;
(r) contact area of the test probe and sample;
(s) any combination of the above parameters;
(t) any change in those parameters, or combinations of those parameters, in multiple cycle testing; or
(u) the response of the material to a series or combination of the above measurements.

4. The method of claim 1 in which the test probe is coated with a chemical or biological functionality to interact with the material.

5. The method of claim 1 in which the reference probe is in the form of a sheath in which the test probe is disposed, the end of the reference probe being proximal the test probe tip serving as said reference.

6. The method of claim 1 for characterizing a material in which the test probe is suspended, the suspension being at a stiffness that is greater than the stiffness of the material.

7. An instrument for characterizing a material, comprising:
a housing;
a test probe constructed for insertion at least a micron into the material and withdrawal from the material;
optionally a reference probe constructed either for insertion into the material or to contact another material without insertion;
means for measuring a property of the material related to the test probe's (a) interaction with the material, (b) insertion into the material, (c) movement in the material, or (d) withdrawal from the material.

8. The instrument of claim 7 in which the material property is one or more of:
(a) a mechanical property;
(b) the resistance of the material to microscopic fracture by the test probe;
(c) a curve of the indentation depth into the material versus force needed;
(d) indentation of the material at a fixed force;
(e) indentation of the material at a fixed impact energy;
(f) the adhesion of the material on the test probe;
(g) the elastic modulus of the material;
(h) the resistance of the material to fatigue fracture;
(i) the resistance to penetration of a screw into the material;
(j) the rotary friction on the material;
(k) a curve of the indentation depth vs. time after an impact;
(l) a curve of the force vs. time after impact to set distance;
(m) curves of the indentation depth vs. time for repetitive cycles or impacts;
(n) maximum indentation force;
(o) maximum indentation distance;
(p) energy dissipated during the indentation and retraction cycle or an impact;
(q) adhesion force during retraction;
(r) contact area of the test probe and sample;
(s) any combination of the above parameters;
(t) any change in those parameters, or combinations of those parameters, in multiple cycle testing; or
(u) the response of the material to a series or combination of the above measurements.

9. The instrument of claim 7 in which the test probe is coated with a chemical or biological functionality to interact with the material.

10. The instrument of claim 7 in which the reference probe is in the form of a sheath in which the test probe is disposed, the end of the reference probe being proximal the test probe tip serving as said reference.

11. The instrument of claim 7 in which the test probe is constructed at one end as a dental pick.

12. The instrument of claim 7 including means for suspending the test probe at a stiffness greater than the stiffness of the material.
